(19) United States
(12) Patent Application Publication
(10) Pub. No.: US 2025/0134377 A1
(43) Pub. Date: May 1, 2025

(54) SYSTEM AND METHOD FOR COLLECTING BIOMETRIC DATA GENERATED THROUGH BODYSCANNER FOR HEALTH-RELATED APPLICATIONS

(71) Applicant: PANTHERION HOLDING GMBH, Munich (DE)

(72) Inventor: Armin KESHMIRI, Munich (DE)

(73) Assignee: PANTHERION HOLDING GMBH, Munich (DE)

(21) Appl. No.: 18/836,759

(22) PCT Filed: Feb. 3, 2023
(86) PCT No.: PCT/EP2023/052729

§ 371 Date: Aug. 8, 2024

(30) Foreign Application Priority Data
Feb. 8, 2022 (EP) 22155733.3

(51) Int. Cl. A61B 5/00; A61B 5/117

(52) U.S. Cl. CPC A61B 5/0064; A61B 5/0015; A61B 5/117

(57) ABSTRACT

A method and a system assisting in monitoring the health condition of persons. A security body scanner is configured for a scan of a body of a person for security reasons with the further feature of comprising a communication component that is configured for providing health data of the person on the basis of the body scan.

### FIELD

The invention, according to the various embodiments described herein, relates to systems and methods for scanning people, for example, people in security, and to make further use of the data for health-related purposes.

### BACKGROUND

The use of radiation in the range from approximately 10 gigahertz (GHz) to approximately 100 GHz in non-destructive material testing and for full-body scanners is already known. The corresponding millimeter waves or respectively microwaves are suitable, for example, for the detection of metallic, but especially also non-metallic objects, for example, objects made from ceramic materials or certain synthetic materials. At the same time, air and materials such as those used, for example, for clothing, are transparent for such radiation. Accordingly, millimeter waves or microwaves are suitable, for example, for contactless detection of objects concealed beneath the clothing. Corresponding scanners can supplement or replace metal detectors, for example, in the field of security checks at airports or security relevant public facilities. During the operation of such a scanner, a microwave signal with a given bandwidth is generated and transmitted from at least one antenna in the direction towards a human body, optionally with objects concealed beneath the clothing. The microwave signals are reflected from the body and from the concealed objects. The reflections are received by at least one antenna. Separate transmitting and receiving antennas can be provided. A scanner can also include a plurality of transmitting and/or receiving antennas. Such a scanner can be operated, for example, in a multi-static mode, in which a transmitting antenna transmits a signal, and reflections of this signal are received according to amplitude and phase position by several receiving antennas or by all receiving antennas, after which the next transmitting antenna transmits a signal etc.

According to Wikipedia in February 2022 a full-body scanner is a device that detects objects on or inside a person's body for security screening purposes, without physically removing clothes or making physical contact. As an example, those scanners are offered by Rohde & Schwarz with the designation Quick Personnel Security Scanner (QPS). Depending on the technology used, the operator may see an alternate-wavelength image of the person's naked body, merely a cartoon-like representation of the person with an indicator showing where any suspicious items were detected or full X-ray image of the person. For privacy and security reasons, the display is generally not visible to other passengers, and in some cases is located in a separate room where the operator cannot see the face of the person being screened. Unlike metal detectors, full-body scanners can detect non-metal objects, which became an increasing concern after various airliner bombing attempts in the 2000s. Transmission X-ray body scanners can also detect swallowed items or hidden in body cavities of a person.

Starting in 2007, full-body scanners started supplementing metal detectors at airports and train stations in many countries.

Three distinct technologies have been used, though the use of backscatter X-ray has now been discontinued in many countries:
Millimeter wave scanners use non-ionizing electromagnetic radiation similar to that used by wireless data transmitters, in the Extremely High Frequency (EHF) radio band (which is a lower frequency than visible light). The health risks posed by these machines are still being studied, and the evidence is mixed, though millimeter wave scanners do not generate ionizing radiation.
Backscatter X-ray machines use low dose penetrating radiation for detecting suspicious metallic and non-metallic objects hidden under clothing or in shoes and in the cavities of the human body. Considerable debate regarding the security of this method sparked investigations, ultimately leading multiple countries to ban the usage of them.
Transmission (Penetrating) X-ray security scanners. In these scanners, X-ray radiation passes through the human body and then is captured by a detector or array of detectors. This type of full body scanners allows to detect objects hidden not only under the clothes, but also inside the human body (for example, drugs carried by drug couriers in the stomach) or in natural cavities. The dose received is usually not higher than 0.25 μSv and is mainly regulated by the American radiation security standard for personal search systems using gamma or X-ray radiation ANSI 43.17.2009. Due to the low dose, the resulting image could not have a diagnostic quality for medical purposes but is quite enough to detect items not characteristic for human biology which could be a contraband.

For the use of the present application one or more of the before technologies are being made use of what is further called a “security body scanner”.

Methods for aperture synthesis can also be used, for example, methods of digital beam forming (“Digital Beamforming”, DBF), in which a focusing of the transmitted and/or received radiation can take place for the scaling of objects without mechanical movement of the antennas or electromagnetic bundling through lenses or similar. Instead, a focusing is implemented only through targeted control of the transmitting antennas and/or evaluation of the signals detected by the receiving antennas. In the latter case, a signal reflected from a given spatial point by a software-based algorithm in the direction towards several antennas and received there, is evaluated accordingly.

Even in the case of optimized hardware and with the use of highly developed signal processing, there is a continuing general requirement for an improvement in the detection capability of such scanners. It should be possible to obtain views or images of people and objects with sufficiently clear contours, so that all automatic detection, for example, of undesirable objects, is possible, and/or so that security personnel can be supported with the most reliable detection possible. In this context, the measurement times should be as short as possible, so that people can be scanned, for example, in an undisturbed movement flow.

Efforts have been directed primarily towards an improvement of illumination. For example, WO 2012/167847 A1 proposes the additional provision of at least one reflector element alongside an arrangement of transmitting/receiving antennas. As a result of the reflector element, a relatively larger proportion of the micrometer-wave signals can be used for the reconstruction.

U.S. Pat. No. 6,965,340 B1 describes a security gate with four panels arranged in an angled am1er. A person walks in a straight line through the gate and is scanned in this context by means of microwave radiation.

All the before mentioned documents are herein incorporated by reference.

### SUMMARY

It is the object underlying the present invention to provide an improved or alleviated system and a method for (further) collecting health-related biometric data.

This object is attained with the system and method as claimed and described.

The present invention relating to a system, a method, software etc. can be also used for test and/or calibration uses without any person or body present.

The present invention relates to a method and a system assisting in a further monitoring of the health condition of persons. It is advantageous to make use of data that is anyhow obtained for other reasons, such as security reasons. In particular, there is provided a security body scanner as known already and established in many security-relevant locations, such as the security check at airports. The security body scanner is configured for a scan of a body of a person for security reasons with the further feature of comprising a communication component that is configured for providing health data of the person on the basis of the body scan.

Such scanners are particularly capable of checking and/or any orthopedic problems, such as skeleton-related mispositions, at the spine, the knees etc., and/or any other potential problem that can be spotted at the skin or envelope of a body. Other health issues could be thyroidal and/or abdominal problems or enlargements, outer tumors, or tumors impacting the skin or envelope of a person etc. As the resolution of those security scanners is quite high, the benefit is respectively obvious. Additionally, the generated biometric data can be used according to “big data” valuation and “deep phenotyping”. Means, a certain amount of generated biometric data can generate possibilities of health-related issues or diseases without having obvious signs. The biometric data also can be used to generate fitness and lifestyle related applications to monitor peoples progress in generating body contour using the right training and/or healthcare support. Also, the biometric data can be used for producing tailored clothing. The biometric data also can be used for “gaming” purposes to generate someone's own avatar in a video game. Also generating a person's own NFT (Non-fungible Token) using blockchain technology is a use case of biometric body surface data.

The communication component can be inactive and only be configured to provide the health status data upon a trigger signal. This is the case when a person to be scanned wants to gain the respective findings or to make further use of the health data for his/her own benefit. The trigger signal can be provided by a user, such as the person scanned and/or an operator of the system. The trigger signal can be provided after an identification of the person scanned is made in order to avoid any wrong assignments.

The health data can be provided only after the person scanned has provided the trigger signal and/or after the identification of the person scanned. The communication component can be configured to transmit the health data onto a handheld device (such as a smart phone) of the person scanned. This can be controlled by a software application (app) on that handheld.

The communication component can be configured to transmit the health data to a remote database, such as the cloud and/or any other remote and/or local server. The communication component can be configured to transmit the health data in encrypted form. The encryption can have the side effect to also compress the data but is particularly provided to appropriately handle this sensitive health-data. The encrypted health data can be decrypted with a key provided to the person scanned. This decryption key could be transmitted to the person scanned or could be provided by the software application or in any other manner. The encrypted health data can be decrypted with the key provided to the person scanned on a handheld device, such as a smartphone.

Further an analytics component can be provided that is configured to determine health-related information to already existing health data. It can also be used to monitor a person over time and compare the health-data in a time-resolved manner.

The system can further comprise a database and an analytics component that is configured to determine health-related information in the health data over time with respect to the person scanned. The analytics component may be provided in a manner to better concentrate on any deviations to normal or previous scans etc. The analytics component cab be configured to be trained by aggregated health data from other persons over time and can even be trained by respective training data. The analytics component can be configured for determining health related abnormalities based on existing data of a large number of persons.

The analytics component can be configured for determining health related abnormalities based on existing data of a large number of persons and a probability approach. Further, a feeding component can be provided that is configured to feed the health data and/or health-related information to a health database assigned to the person scanned.

Moreover, a transmitter can be provided for transmitting the radiation and a receiver for receiving the radiation from an object, such as a body of a person. The transmitter can transmit radiation in the range 70 Ghz to 80 GHz. Other frequencies can be also used.

As basically known, there are two opposing transmitters with implemented receivers in the same panel. Other embodiments are included as well. The transmitters can transmit radiation with different frequencies.

Panels each can comprise transmitters and receivers, preferably between 10 and 500, more preferably between 50 and 200, and more preferably between 100 and 150.

A detector can be further arranged for contactless detection of objects concealed beneath the clothing. The detector can be integrated with the communication component and can be modularly arranged. An information display can be arranged for an operator of the system.

A user terminal can be configured for a communication with the person to be scanned and with the functionalities mentioned before and below.

The user-terminal can comprise a short-range communication component configured to communicate with a handheld device of the person to be scanned, such as NFC, Bluetooth etc.

The user-terminal can be configured for providing and/or processing a decryption-key and/or an ID (identifier) to the handheld device of the person to be scanned.

The system can further comprise a first user-terminal upstream and a second user-terminal (6) downstream a body scan, wherein the first user-terminal (5) is configured for triggering the system to provide health data and/or for transferring a decryption key to a handheld device of the person to be scanned after the scan, and the second user-terminal being configured to transfer the respective information onto the handheld device of the person scanned.

Below is a list of system embodiments. Those will be indicated with a letter “S”. Whenever such embodiments are referred to, this will be done by referring to “S” embodiments.
S1. System for a security body scanner wherein the security body scanner is configured for a scan of a body of a person for security reasons further comprising a communication component (3) that is configured for providing health data and/or biometric data for of the person on the basis of the body scan.
S2. System according to the preceding system embodiment wherein the communication component (3) is configured to provide the health status data upon a trigger signal.
S3. System according to the preceding system embodiment wherein the trigger signal is provided by a user, such as the person scanned and/or an operator of the system.
S4. System according to any of the two preceding system embodiments wherein the trigger signal is provided after an identification of the person scanned.
S5. System according to any of the preceding system embodiments wherein the health data is provided only after the person scanned has provided the trigger signal and/or after the identification of the person scanned.
S6. System according to any of the preceding system embodiments wherein communication component (3) is configured to transmit the health data onto a handheld device of the person scanned.
S7. System according to any of the preceding system embodiments wherein the communication component (3) is configured to transmit the health data to a remote database, such as the cloud (4).
S8. System according to the preceding system embodiment wherein the communication component (3) is configured to transmit the health data in encrypted form.
S9. System according to the preceding system embodiment wherein the encrypted health data can be decrypted with a key provided to the person scanned.
S10. System according to the preceding system embodiment wherein the encrypted health data can be decrypted with a key provided to the person scanned on a handheld device, such as a smartphone.
S11. System according to any of the preceding system embodiments further comprising an analytics component (4) that is configured to determine health-related information in the health data.
S12. System according to any of the preceding system embodiments further comprising a database and an analytics component (4) that is configured to determine health-related information in the health data over time with respect to the person scanned.
S13. System according to any of the two preceding system embodiments wherein the analytics component (4) is configured to be trained by aggregated health data.
S14. System according to any of the three preceding system embodiments wherein the analytics component (4) is configured for determining health related abnormalities based on existing data of a large number of persons.
S15. System according to any of the three preceding system embodiments wherein the analytics component (4) is configured for determining health related abnormalities based on existing data of a large number of persons and a probability approach.
S16. System according to any of the preceding system embodiments further comprising a feeding component that is configured to feed the health data and/or health-related information to a health database assigned to the person scanned.
S17. System according to any of the preceding system embodiments further comprising a transmitter (1,2) for transmitting the radiation and a receiver (1,2) for receiving the radiation from an object, such as a body of a person.
S18. System according to the preceding system embodiment wherein the transmitter (1,2) transmit radiation in the range 70 Ghz to 80 GHz.
S19. System according to any of the two preceding system embodiments with two opposing transmitters (1,2) with implemented receivers (1,2).
S20. System according to any of the three preceding system embodiments wherein the transmitters (1,2) transmit radiation with different frequencies.
S21. System according to any of the four preceding system embodiments wherein panels (1,2) each comprise transmitters and receivers, preferably between 10 and 500, more preferably between 50 and 200, and more preferably between 100 and 150.
S22. System according to any of the preceding system embodiments further comprising a detector (3) for contactless detection of objects concealed beneath the clothing.
S23. System according to the preceding system embodiment wherein the detector (3) is integrated with the communication component (3).
S24. System according to any of the preceding embodiments further comprising an information display (6) for an operator of the system.
S25. System according to any of the preceding embodiments further comprising a user terminal (5) configured for a communication with the person to be scanned.
S26. System according to the preceding embodiment wherein the user-terminal comprises a short-range communication component configured to communicate with a handheld device of the person to be scanned.
S27. System according to the preceding embodiment wherein the user-terminal (5) is configured for providing a decryption-key and/or an ID (identifier) to the handheld device of the person to be scanned.
S28. System according to any of the preceding embodiments wherein the system comprises a first user-terminal (5) upstream and a second user-terminal (6) downstream a body scan, wherein the first user-terminal (5) is configured for triggering the system to provide health data and/or for transferring a decryption key to a handheld device of the person to be scanned after the scan, and the second user-terminal (6) being configured to transfer the respective information onto the handheld device of the person scanned.

Below is a list of system use embodiments. Those will be indicated with a letter “SU”. Whenever such embodiments are referred to, this will be done by referring to “SU” embodiments.
SU 1. Use of a system according to any of the preceding system embodiments for providing health data and/or biometric data and/or phenotyping data and/or health-related data of a person scanned for security reasons.
SU 2. Use of a system according to any of the preceding system embodiments for diagnosing any health-related modification of a body of a person over time.
SU 3. Use of a system according to any of the preceding system embodiments for diagnosing any health-related modification of orthopedic and/or organic and/or tissue-related form.

Below is a list of method embodiments. Those will be indicated with a letter “M”. Whenever such embodiments are referred to, this will be done by referring to “M” embodiments.
M1. A method of operating a system according to any of the preceding system embodiments.
M2. A method for scanning bodies of a person for security reasons with the further step of gaining health-related data of the person.
M3. A method for operating a full-body scanner for security reasons with the further step of gaining health-related data of the person scanned.

Below is a list of method use embodiments. Those will be indicated with a letter “MU”. Whenever such embodiments are referred to, this will be done by referring to “MU” embodiments.
MU 1. Use of a method according to any of the preceding method embodiments for providing health data or health-related data and/or biometric data and/or phenotyping data and/or of a person scanned for security reasons.
MU 2. Use of a method according to any of the preceding method embodiments for diagnosing any health-related modification of a body of a person over time.
MU 3. Use of a method according to any of the preceding method embodiments for diagnosing any health-related modification of orthopedic and/or organic and/or tissue-related form.

Below is a list of computer-implemented embodiments. Those will be indicated with a letter “C”. Whenever such embodiments are referred to, this will be done by referring to “C” embodiments.
C1. A computer program product comprising instructions which, when the program is executed by a computer, cause the computer to carry out the method according to the preceding method embodiments.
C2. A software application (app) making use of the computer program product according to C1.

### BRIEF DESCRIPTION OF THE DRAWINGS

Further potential and thus non-limiting features, details and advantages of the invention will be discussed in the drawings are shown clearly.

FIG. 1 shows an example of components in accordance with the present invention.

### DETAILED DESCRIPTION

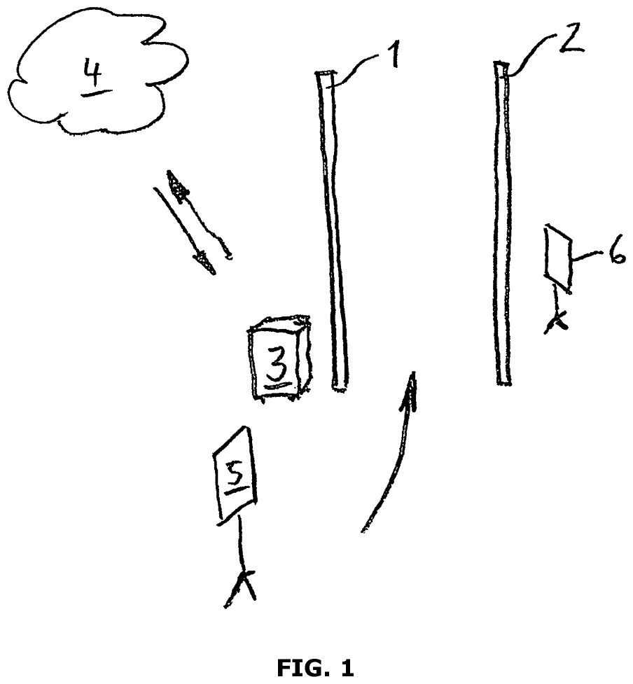

FIG. 1 shows an example of a security station as it could be positioned at an airport security check etc. The transmitters 1, 2 are known as such. A terminal 6 is also usually present for an operator who is checking the scanning results of persons walking into the scanning area between the transmitters 1, 2 and being scanned in between them for a short period of time, such as 1-4 seconds. This is an example only. Usually, just avatars of the people are shown with dubious pieces sketched on that avatar picture.

It is now a gist of the present invention to make additional use of the scanning results for health monitoring purposes. For that, there is provided a communication component 3 that is configured to communicate the scanning result either in a further unprocessed form or with a further processed form as additional health information. The health data could be communicated to a remote server, such as the so-called cloud 4, and/or to a local server and/or to a device in the possession of the person scanned, such as a handheld (not shown).

The health data can be stored or stored in encrypted manner and/or communicated to the person's handheld for further use. A use could be that the information is fed or merged with health data on a respective server under the so-called e-health concept etc. This could be also triggered by the person, particularly with the health data on a handheld. There could be provided a respective software application (app).

It may not be obligatory to make use of that health data but triggered by the person to be scanned. For that reason and to identify that person, there is shown a first terminal 5 upstream of the walking route of a person to be scanned where he/she can identify herself/himself and/or trigger the further use of the scanning results for health reasons.

It is to be noted that other embodiments with further different arrangements of structural components are covered by the present invention.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be the preferred order, but it may not be mandatory to carry out the steps in the recited order. That is, unless otherwise specified or unless clear to the skilled person, the orders in which steps are recited may not be mandatory. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1. A system for a security body scanner wherein the security body scanner is configured for a scan of a body of a person for security reasons further comprising a communication component that is configured for providing health data for of the person on the basis of the body scan.

2. The system according to claim 1 wherein the communication component is configured to provide the health status data upon a trigger signal.

3. The system according to claim 2 wherein the trigger signal is provided by a user, such as the person scanned and/or an operator of the system.

4. The system according to claim 2 wherein the trigger signal is provided after an identification of the person scanned.

5. The system according to claim 2 wherein the health data is provided only after the person scanned has provided the trigger signal and/or after the identification of the person scanned.

6. The system according to claim 1 wherein communication component is configured to transmit the health data onto a handheld device of the person scanned.

7. The system according to claim 1 wherein the communication component is configured to transmit the health data to a remote database, such as the cloud.

8. The system according to claim 7 wherein the communication component is configured to transmit the health data in encrypted form.

9. The system according to claim 8 wherein the encrypted health data can be decrypted with a key provided to the person scanned.

10. The system according to claim 9 wherein the encrypted health data can be decrypted with a key provided to the person scanned on a handheld device, such as a smartphone.

11. The system according to claim 1 further comprising an analytics component that is configured to determine health-related information in the health data, wherein the analytics component is optionally configured to be trained by aggregated health data.

12. The system according to claim 1 further comprising a database and an analytics component that is configured to determine health-related information in the health data over time with respect to the person scanned, wherein the analytics component is optionally configured to be trained by aggregated health data.

13. (canceled)

14. A method of operating a The system according to claim 1.

15. A method for scanning bodies of a person for security reasons with the further step of gaining health-related data of the person.

16. A method for operating a full-body scanner for security reasons with the further step of gaining health-related data of the person scanned.

17. A computer program product comprising instructions which, when the program is executed by a computer, cause the computer to carry out the method according to claim 14.

18. The The system according to claim 11 wherein the analytics component is configured for determining health related abnormalities based on existing data of a large number of persons.

19. The The system according to claim 1 further comprising a feeding component that is configured to feed the health data to a health database assigned to the person scanned.

20. The The system according to claim 1 further comprising a user terminal configured for a communication with the person to be scanned, wherein the user-terminal comprises a short-range communication component configured to communicate with a handheld device of the person to be scanned, wherein the user-terminal is configured for providing a decryption-key and/or an ID (identifier) to the handheld device of the person to be scanned.

21. The The system according to claim 1 wherein the system comprises a first user-terminal upstream and a second user-terminal downstream a body scan, wherein the first user-terminal is configured for triggering the system to provide health data and/or for transferring a decryption key to a handheld device of the person to be scanned after the scan, and the second user-terminal being configured to transfer the respective information onto the handheld device of the person scanned.
